(19) United States
(12) Patent Application Publication
(10) Pub. No.: US 2018/0156796 A1
(43) Pub. Date: Jun. 7, 2018

(54) Methods of Detection of Antibiotic-Resistant Bacteria

(71) Applicant: Silver Lake Research Corporation, Azusa, CA (US)

(72) Inventor: Mark Geisberg, Azusa, CA (US)

(73) Assignee: Silver Lake Research Corporation, Azusa, CA (US)

(21) Appl. No.: 15/829,815

(22) Filed: Dec. 1, 2017

Related U.S. Application Data
(60) Provisional application No. 62/430,721, filed on Dec. 6, 2016.

(51) Int. Cl. G01N 33/569

(52) U.S. Cl. CPC G01N 33/56916; G01N 33/56911

(57) ABSTRACT

The invention relates to novel methods of diagnosis of the presence of antibiotic-resistant bacteria in a liquid or solid sample, detection of antibiotic-resistant bacterial infections in humans or animals, and the use of antibodies or other specific binding molecules capable of binding to products of reactions carried out by bacterial enzymes which confer antibiotic resistance upon bacteria.

### CROSS-REFERENCE TO RELATED APPLICATIONS

This application is claims the benefit of U.S. Provisional Patent Application No. 62/430,721, filed on Dec. 6, 2016, the contents of which are incorporated herein by reference in its entirety.

### BACKGROUND

Therapeutic treatment of bacterial infections of humans and animals relies heavily on antibiotics, chemical entities that selectively target the pathogenic bacteria and not the host. Antibiotic treatment fails when the infecting bacteria are resistant to the antibiotic. Such failure of treatment results not only in deleterious outcomes for the patient, but also in the potential proliferation of the resistant pathogen due to favorable growth conditions and further opportunity for infection of other hosts. Rates of resistance to commonly used antibiotics are increasing worldwide, with many once-effective drugs no longer recommended in many parts of the world. It is therefore desirable to identify the presence or absence of resistance to a particular antibiotic in an infecting bacterial pathogen, especially advantageous when the resistance can be identified before the antibiotic is dispensed.

The oldest, most commonly used class of antibiotics is the β-lactams, which include penicillin and its derivatives, cephalosporins, and carbapenems. However, in the US, the rate of resistance to penicillins in common bacterial infections exceeds 30%. These antibiotics have been eliminated from the usable therapeutic arsenal, as major guidelines for usage of antimicrobials proscribe the dispensing of a β-lactam antibiotic if the expected prevalence of resistance exceeds 20%. The overwhelming majority of β-lactam resistance can be attributed to the expression of bacterial enzymes called β-lactamases, which directly inactivate the drug molecule by cleaving the lactam ring. Over 1000 β-lactamases have been described, with great variation in the range of β-lactams they can deactivate.

Current methods to diagnose the presence of antibiotic resistance in bacteria include laboratory culture of samples to determine growth of the infectious agent on nutrient medium in the presence of specific antibiotics, and the detection of bacterial DNA sequences associated with antibiotic resistance by polymerase chain reaction (PCR) or other methods. Major drawbacks of these methods include the laborious nature of the techniques, the need for specialized equipment and a laboratory environment, and the time required to obtain a result. Most techniques also require a sample to be collected and transported to a laboratory capable of performing the test, adding additional time to obtain a result. Culture-based antibiotic susceptibility assays remain the gold standard in the identification of antibiotic resistance; however, the technique typically requires several days to obtain a result. Detection of antibiotic resistance by PCR necessitates the selection and use of specific DNA sequences from a large number of resistance-associated biomarkers, e.g., β-lactamases, that may be present. Because of these drawbacks, testing for antibiotic resistance is rarely performed before antibiotics are dispensed, resulting in non-cures for patients with resistant infections and further selection of the resistance trait by inappropriate antibiotic use.

Rapid assays for the detection of specific β-lactamases also have been described. These assays detect the β-lactamase protein using antibody binding. However, these methods are of limited clinical use because of the wide variety of extant β-lactamases, of which only a few are detected by the assay.

Assays for the detection of β-lactamase activity are also known. The colorimetric nitrocefin assay detects β-lactamase activity through the conversion of the yellow nitrocefin substrate to the red product by these enzymes. However, the nitrocefin assay requires a large number of bacteria to be present, and is typically used only after culture and identification of the pathogenic bacteria. Furthermore, quantitation of the assay requires a spectrophotometer. Even with quantitation, is not possible to determine the level of resistance, e.g., whether the resistance of the given pathogen to a particular antibiotic is enough to confer the ability to survive treatment of the infection by the antibiotic. Additional assays to detect the products of β-lactamase activity have been described (Lee W S, et al. J Clin Microbiol. 1981 January; 13(1):224-5; Skinner A, et al. J Clin Pathol. 1977 November; 30(11):1030-2; Chen K C, et al. J Clin Microbiol. 1984 June; 19(6):818-25). However, none of the assays have been found appropriate for widespread use due to a variety of reasons, including time-to-result limitations, sensitivity, and interference from biological matrices.

There remains a need for simple and rapid methods to detect the trait of antibiotic resistance in bacteria in a sample, without the need for culture.

### SUMMARY

Embodiments of this invention are directed generally to the field of detection of antibiotic resistance in bacteria. In certain aspects, the invention is directed to methods for detecting the presence of bacteria resistant to specific subsets of aminoglycoside or β-lactam antibiotics. In other aspects, the invention is directed to methods for detecting the presence of bacteria resistant to polymyxin antibiotics.

The present invention includes an embodiment directed to a method of detecting the presence of antibiotic-resistant bacteria in a sample. The method includes the first step of contacting the sample with a substance capable of being a substrate for one or more bacterial enzymes, where the bacterial enzymes are capable of conferring antibiotic resistance upon bacteria possessing the enzymes. In the second step, one or more antibodies, antibody fragments, or aptamers are used to detect the presence of one or more products of enzymatic reactions carried out by the bacterial enzymes upon the substance. The detection of at least one or more products of enzymatic reactions indicates the presence in the sample of bacteria resistant to one or more antibiotics. In one aspect, the one or more bacterial enzymes are β-lactamases, and the substance is an antibiotic or fragment thereof containing a β-lactam moiety such as, for example, a penam moiety. In another aspect, the sample can be treated with one or more substances, such as, for example, a buffer or a detergent, to enhance the activity of the bacterial enzymes. In another aspect, the bacterial enzymes can comprise one or more members of the set of β-lactamases of the carbapenemase class, and the substance can be an antibiotic or fragment thereof containing a carbapenem moiety. In another aspect, the bacterial enzymes can comprise one or more members of the group comprising N-acetyltransferases, O-adenosyletransferases, and O-phosphotransferases, and the substance can be one or more molecules containing amino-sugars capable of being acetylated by bacterial N-acetyltransferases or one or more molecules containing sugars capable of being adenosylated or phosphorylated by bacterial O-adenosyletransferases or O-phosphotransferases. In yet another aspect, the bacterial enzymes are capable of adding ehanolamine, aminoarabinose, or galactosamine to bacterial lipopolysaccharides or to bacterial lipid A. The antibody can be polyclonal or monoclonal. The sample can be a diluted or non-diluted sample of a group comprising urine, blood, serum, blood products, plasma, saliva, bodily fluid, water, culture medium, petroleum product, fuel, liquid undergoing fermentation, or a beverage. The sample can be from human or animal tissue, stool, sputum, or expectorate, or the sample can be an agricultural product, food, solids collected by centrifugation or filtration, soil, or sediment. In one aspect, the detecting can be performed by a competitive immunoassay, an enzyme-linked immunosorbent assay (ELISA), an immunofluorescence assay (IFA), a radioimmunoassay (RIA), a chemiluminescence immunoassay (CLIA), a lateral flow chromatographic test, a dot blot, a chromatographic test, a Western blot, an immunoprecipitation assay, or a lateral flow immunoassay device. In another aspect, the antibodies, antibody fragments, or aptamers are labeled with, for example, biotin, an enzyme, a latex particle, a metal colloid particle, a fluorescent dye, a quantum dot, or carbon nanotube.

In another embodiment of the invention, a kit for detecting the presence of bacteria resistant to one or more antibiotics in a sample is provided. The kit comprises an antibody, an antibody fragment, or aptamer capable of binding one or more products of enzymatic reactions carried out by bacterial enzymes upon a substance capable of being a substrate of the bacterial enzymes. The bacterial enzymes are capable of conferring antibiotic resistance upon the bacteria possessing the enzymes. In one aspect, the kit can comprise a lateral flow chromatographic assay. In another aspect, the kit can have a negative control, a positive control, or both a negative and positive control. In one aspect, the kit can further comprising a solid substrate, wherein the antibody, antibody fragment is immobilized on the surface of the solid substrate. The solid substrate can comprise a particle, a bead, a plastic or glass surface, a porous membrane, an array, or a chip.

In another embodiment, the present invention describes a method of treating bacterial infection in an individual using the methods described above on a sample from the individual, resulting in detection of antibiotic resistance in the sample. From the results of the method, an antibiotic is chosen, thereby treating the bacterial infection in the individual.

### DRAWINGS

These and other features, aspects, and advantages of the present invention will become better understood with regard to the following description, appended claims, and accompanying drawings, where:

FIG. 1 depicts a graph showing the results of a competitive ELISA of samples containing beta-lactam-resistant and beta-lactam-sensitive R coil; and

FIGS. 2A-2E depict the chemical structures referred to herein.

### DESCRIPTION

Described herein is an improved strategy for the detection of antibiotic-resistant bacteria. Resistance of bacteria to many antibiotics, including β-lactams and aminoglycosides, can be mediated by enzymes that modify or inactivate these antibiotics. The present invention consists of assays and a method for detection of antibiotic-resistant bacteria based on the use of immunoassays for the products of reactions produced by bacterial enzymes which confer antibiotic resistance.

Also described are assays and methods that can be used to rapidly detect antibiotic-resistant bacteria in a sample, as well as uses of these assays in a variety of settings, including, but not limited to, testing of patient samples to detect the presence of antibiotic-resistant bacterial infection in the patient, and the selection of appropriate antibiotics for treatment. The presence of certain enzymes, including but not limited to β-lactamases and aminoglycoside-modifying enzymes, confers resistance to a wide range of antibiotics that can be modified and deactivated by these enzymes. The detection of these enzymes in a sample indicates the likely presence in the sample of bacteria resistant to any antibiotic that can be modified by the detected enzyme. The presence of other enzymes, including those capable of adding amine-containing moieties onto the bacterial lipid A structure, confers resistance to colistin and other polymyxin antibiotics. The detection of these enzymes in a sample indicates the likely presence in the sample of bacteria resistant to polymyxins.

A particularly effective method of detecting antibiotic-resistant bacteria in a sample is to a) contact the sample with a substance that can be a substrate for one or more enzymes of interest; b) assay for the products of modification of the provided substance by the enzyme(s) of interest using antibodies, or similar molecules, that specifically bind the products; c) infer the presence or absence of enzymes of interest from the presence or absence of reaction products mediated by these enzymes; d) infer the presence or absence of bacterial resistance to specific antibiotics from the known reactivity profiles of detected or non-detected enzymes of interest. The detection or non-detection of specific antibiotic resistance in a sample can then guide the choice of antibiotics used for treatment of bacterial infections. The potential completion of the described detection method through the use of an immunoassay can avoid the use of treatment with antibiotics to which the targeted bacterial infection is resistant, and/or the selection of antibiotics to which the targeted bacterial infection is susceptible.

The terms “a,” “an,” and “the” and similar referents used herein are to be construed to cover both the singular and the plural unless their usage in context indicates otherwise.

As used herein, the term “comprise” and variations of the term, such as “comprising” and “comprises,” are not intended to exclude other additives, components, integers or steps.

“Enzyme” as used herein is a biological molecule capable of converting one substance, termed a substrate, into a different substance, termed a product.

“Resistance” as used herein is a measurable characteristic indicative of the ability of bacteria to survive treatment with a drug. More specifically, antibiotic resistance is a measurable characteristic indicative of the ability of bacteria to survive treatment with one or more drugs used for treating bacterial infections.

“Substrate” as used herein is any substance that can be converted by a particular enzyme into a different substance. Those of skill in the art will be able to select existing substances or design new substances which are substrates for a given enzyme or set of enzymes.

“Product” as used herein is a substance resulting from the action of an enzyme upon a substrate.

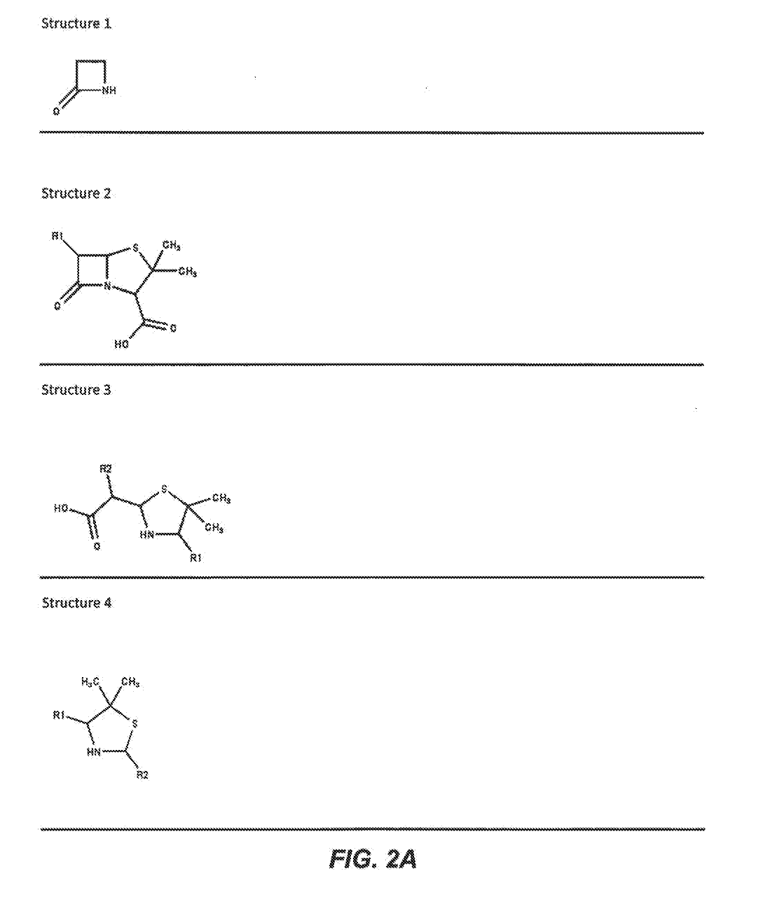

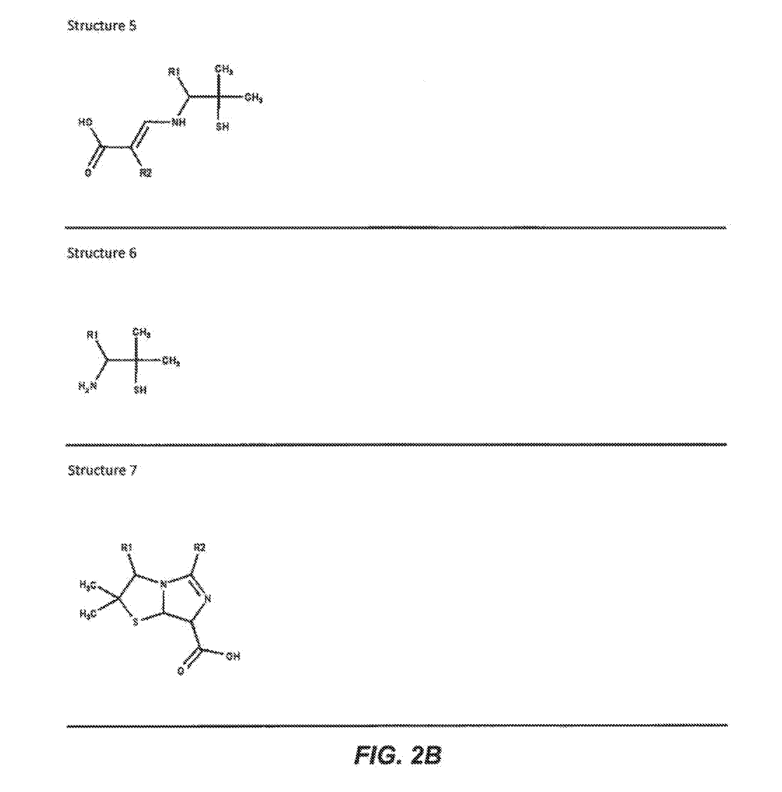

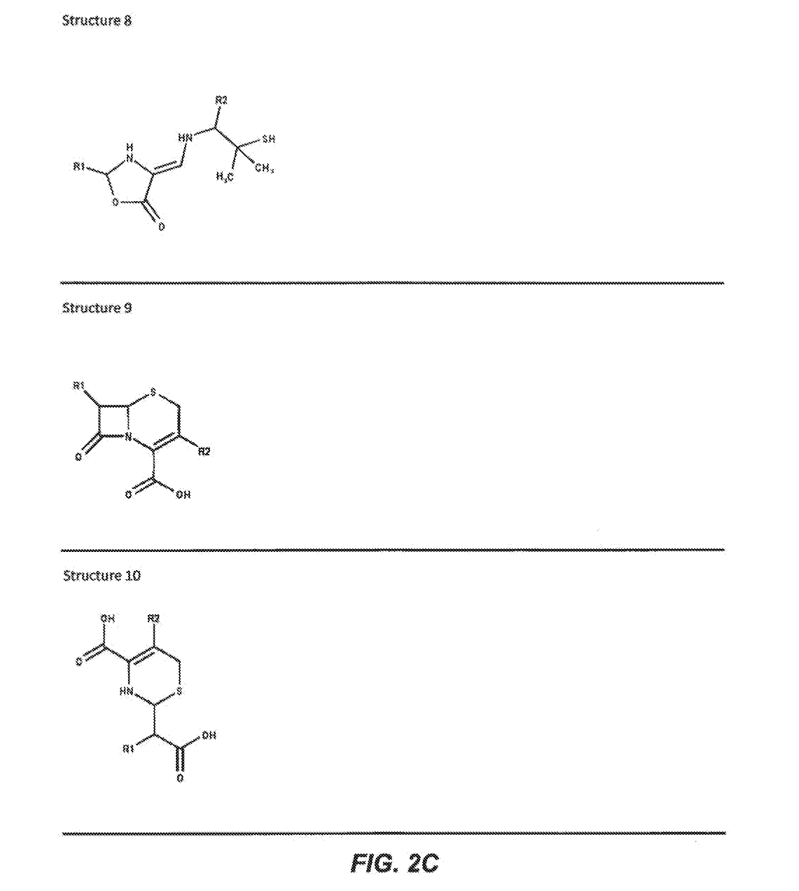

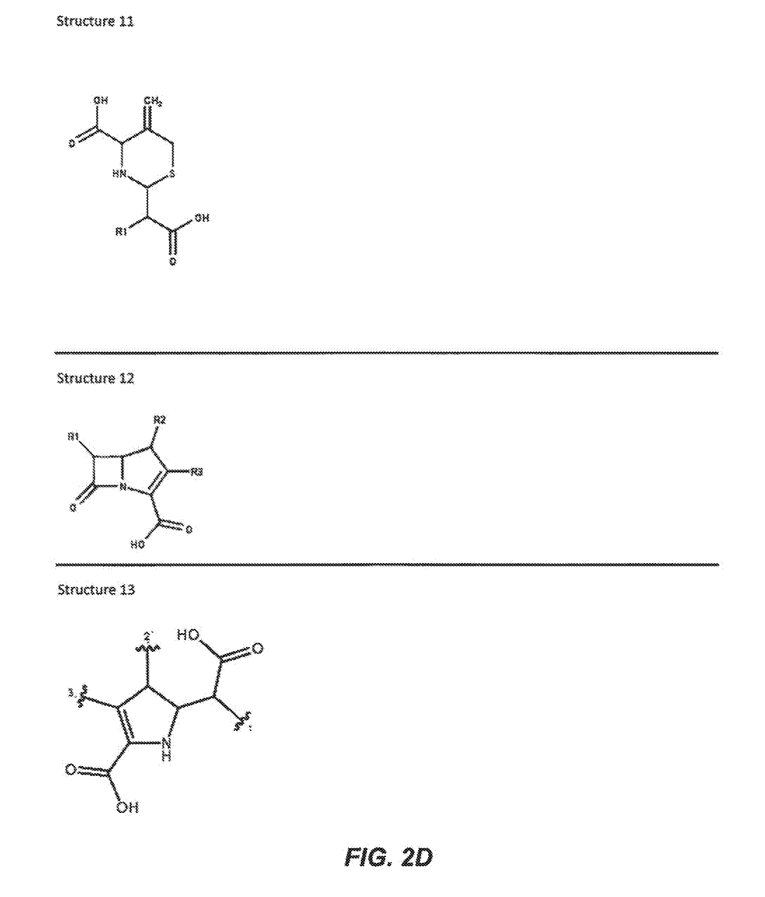

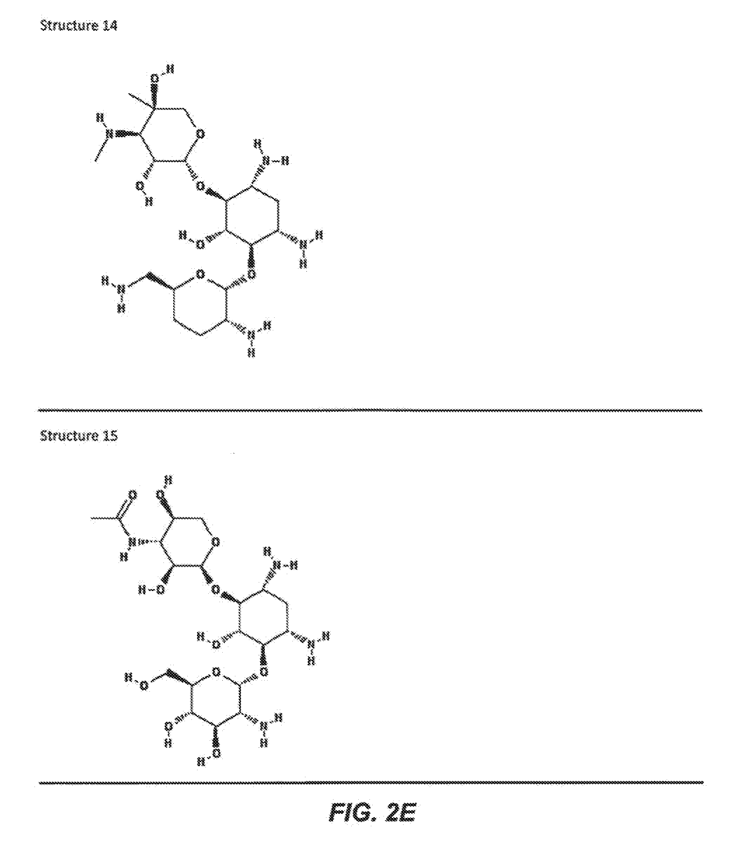

“β-lactam” as used herein is a substance comprising 2-azetidinone (structure 1, FIG. 2A), whether by itself or as part of a larger structure, and is also consistent with the meaning of “a substance which possesses (structure 1, FIG. 2A).

“β-lactamase” as used herein is an enzyme capable of converting a β-lactam into a different substance.

An “antibody” is an immunoglobulin which possesses the ability to combine with an antigen. It comprises at least two heavy (H) chains and two light (L) chains inter-connected by disulfide bonds. Non-limiting examples of antibodies include monoclonal antibodies (e.g., full length or intact monoclonal antibodies), polyclonal antibodies, multivalent antibodies, and multispecific antibodies. An antibody can be affinity-matured. The methods for producing, screening and characterizing antibodies have been described and are known by those of skill in the art. An antibody can include intact immunoglobulin molecules, fragments of immunoglobulins, aptamers, and polypeptides that have been engineered to have an antibody-like binding site, which are capable of binding an epitope of any type of target molecule. Any type of antibody known in the art can be generated to bind specifically to a product of enzymatic action upon a substrate.

The term “antibody fragment” refers to a portion of an intact antibody, wherein the portion preferably retains at least one, preferably most or all, of the functions normally associated with that portion when present in an intact antibody. Examples of antibody fragments include Fab, Fab′, F(ab′)₂, and Fv fragments, linear antibodies, single-chain antibody molecules, and multispecific antibodies formed from antibody fragments.

An “isolated” or “purified” antibody is one which has been identified and separated or recovered, or both, from a component of its natural environment. Contaminant components of an isolated antibody's natural environment are materials that would interfere with diagnostic uses of the antibody. Non-limiting examples of such contaminants include enzymes, hormones, and other proteinaceous or non-proteinaceous solutes.

The terms “monoclonal antibody” or “monoclonal antibody composition” as used herein refers to a preparation of antibody molecules of single molecular composition. A monoclonal antibody composition displays a single binding specificity and affinity for a particular epitope. The monoclonal antibodies herein specifically include “chimeric” antibodies in which a portion of the heavy or light chain, or both, is identical with or homologous to corresponding sequences in antibodies derived from a particular species or belonging to a particular antibody class or subclass, while the remainder of the chain or chains are identical with or homologous to corresponding sequences in antibodies derived from another species or belonging to another antibody class or subclass, as well as fragments of such antibodies so long as they exhibit the desired biological activity.

“Single-chain Fv” or “scFv” antibody fragments comprise the V_{H} and V_{L} domains of antibody, wherein these domains are present in a single polypeptide chain. Generally, the scFv polypeptide further comprises a polypeptide linker between the V_{H} and V_{L} domains which enables the scFv to form the desired structure for antigen binding.

An “antigen” is a predetermined substance to which an antibody can selectively bind. The target antigen may be polypeptide, carbohydrate, nucleic acid, lipid, hapten or other naturally occurring or synthetic compound.

The term “solution” refers to a composition comprising a solvent and a solute, and includes true solutions and suspensions. Examples of solutions include a solid, liquid or gas dissolved in a liquid and particulates or micelles suspended in a liquid.

The term “specificity” refers to the number of different types of antigens or antigenic determinants to which a particular antigen-binding molecule or antigen-binding protein molecule can bind. The specificity of an antigen-binding protein can be determined based on affinity and/or avidity. The affinity, represented by the equilibrium constant for the dissociation of an antigen with an antigen-binding protein (Kd), is a measure for the binding strength between an antigenic determinant and an antigen-binding site on the antigen-binding protein: the lesser the value of the Kd, the stronger the binding strength between an antigenic determinant and the antigen-binding molecule (alternatively, the affinity can also be expressed as the affinity constant (Ka), which is 1/Kd). As will be clear to one of skill in the art, affinity can be determined depending on the specific antigen of interest. Avidity is the measure of the strength of binding between an antigen-binding molecule and the antigen. Avidity is related to both the affinity between an antigenic determinant and its antigen binding site on the antigen-binding molecule and the number of pertinent binding sites present on the antigen-binding molecule. Specific binding of an antigen-binding protein to an antigen or antigenic determinant can be determined by any known manner, such as, for example, Scatchard analysis and/or competitive binding assays, such as radioimmunoassays (RIA), enzyme immunoassays (EIA) and sandwich competition assays.

Treatment of bacterial infections may fail when the infecting pathogen has acquired mechanisms which render it insensitive to the antibiotic drug being used. Bacterial resistance to antimicrobials, or AMR, is a worldwide public health issue. However, rapid and timely detection of antimicrobial resistance in most bacterial infections is currently inadequate. It can take several days to obtain results by standard culture-based antimicrobial susceptibility testing, which is an inappropriately long time to delay treatment. In many clinical situations, therefore, initial treatment is empirical administration of antimicrobial agents. In empirical treatment, failure is common, as well as the further selection of drug resistance. Community-acquired urinary tract infections (CA-UTIs) provide a key example, as these infections are often treated empirically.

Public health agencies and medical organizations, such as the Infectious Disease Society of America (IDSA), issue guidelines designed to limit the spread of AMR. These include limitations on the use of drugs for which local prevalence of resistance exceed a certain level, usually 10-20%. The prevalence of AMR to many antibiotics already exceeds these limits in much of the world, leading to the phasing out of these once-effective drugs in locations where such guidelines are followed. In the US, the older β-lactams, including penicillins, amoxicillin, ampicillin, and cephalosporins, are no longer used as first-line therapies for many infections including CA-UTIs. Reliance on newer classes of antibiotics for first-line treatment has led to increasing prevalence of AMR to these drugs as well.

In general, antibiotics work by targeting a particular cellular process necessary for survival or growth of the targeted bacteria. Resistance to antibiotics can be due to two main mechanism categories—inactivation of the antibiotic molecules, typically by enzymes; and protection of the target bacteria, typically by structural modifications of the target of action or by preventing access to the target, e.g., by rapid efflux of the antibiotic. The present invention is aimed at detecting resistance mediated by enzymatic degradation mechanisms, and can also be effective in detecting resistance mediated by some target modification mechanisms.

Since the discovery of penicillin in 1928, many different classes of antibiotics have been discovered and put into clinical use. These structurally similar groups include the β-lactams, aminoglycosides, macrolides, sulfonamides, polymyxins, and others. Some antibiotics are natural products, others are synthetic or semi-synthetic. For most antibiotics, the range of susceptible bacteria and the mechanism of action have been described.

The β-lactams are the most clinically important class of antibiotics. All members of this group have the 4-membered β-lactam ring as the defining common element. Common classes include the early-generation penicillins, including ampicillin and amoxicillin, with the 6-aminopenicillanic acid as the common core element; the cephalosporins, with the 7-aminocephalosporanic acid as the common core element; and the carbapenems, with a 2,3-dihydro-1H-pyrrole group. Other types of β-lactams include penems, cephems, and monobactams. β-lactams are bactericidal and act by inhibiting the synthesis of the peptidoglycan layer of bacterial cell walls. β-lactams are effective against many Gram-negative and Gram-positive bacteria. Bacterial resistance to β-lactams is typically mediated by β-lactamase enzymes, which destroy the 4-membered β-lactam ring, and less commonly by structural modifications of the target of β-lactam action, the penicillin-binding proteins (PBPs).

Aminoglycoside antibiotics share as a common structure at least one amine-modified sugar (glycoside). Members of this class include streptomycin, kanamycin, and gentamicin. Aminoglycosides act by inhibiting protein synthesis, and are effective against many Gram-negative bacteria. Resistance to aminoglycosides is most commonly mediates by enzymes that modify the drugs, rendering them ineffective. Over 50 such aminoglycoside-modifying enzymes (AMEs) have been described.

The polymyxin class of antibiotics, which includes colistin, has a common structure of a cyclic peptide with a hydrophobic tail. Polymyxins are effective against Gram-negative (GN) bacteria. Polymyxins act by binding to bacterial lipopolysaccharide and disrupting the bacterial cell membrane through interactions with phospholipids. Bacterial resistance to polymyxins is commonly mediated by the enzymatic modification of the lipopolysaccharide, typically by addition of positively charged amine groups, reducing or eliminating the binding of polymyxins.

Many other examples of enzyme-dependent AMR mechanisms are known. Bacterial enzymes capable of conferring resistance to various antibiotics include macrolide esterases; epoxidases degrading fosfomycin-type antibiotics; acetyltransferases capable of inactivating chloramphenicol and streptogamin; kinases and phosphotransferases capable of inactivating macrolides, tuberactinomycins, rifampin and many other types of antibiotics; nucleotidyltransferases capable of inactivating aminoglycosides and lincosamidines; redox enzymes inactivating tetracyclines; and others. These enzymes have been described in many types of antibiotic-resistant bacteria. All such examples can be potentially detected by embodiments of the present invention.

β-lactamases are enzymes that hydrolyze the 4-membered lactam ring of β-lactam antibiotics, thereby deactivating the drug. β-lactamases are the major cause of β-lactam resistance in GN bacteria. There are over 1000 known β-lactamases, classified by structural similarity as well as functional characteristics.

Most β-lactamases are encoded chromosomally by the bacteria that harbor the intact, functional gene. Others are encoded on plasmids and are known to be exchanged between bacteria, even of different species, leading to horizontal transmission of resistance. In most cases, β-lactamases are expressed constitutively, while some are inducible when a β-lactam is present.

The TEM and SHV classes of β-lactamases are some of the most common and clinically important. These enzymes can hydrolyze penicillins and most cephalosporins, but not carbapenems. The carbapenemases, which include the KPC and NDM classes, as well as VIM, IMP, and OXA-48, can hydrolyze all β-lactams including carbapenems. The activity of some β-lactamases, including TEM and SHV but not CTX-M, can be blocked by clavulanic acid, which is a common addition to antibiotic therapy. All β-lactamases can hydrolyze the first-generation penicillin derivatives, including ampicillin and amoxicillin with the common 3,3-dimethyl-7-oxo-4-thia-1-azabicyclo[3.2.0]heptane-2-carboxylic acid structure (structure 2, FIG. 2A). The enzymatic hydrolysis of the common structure results in the initial product, a derivative of penicilloic acid ((5,5-dimethyl-1,3-thiazolidin-2-yl)acetic acid, derivatized at C2, C6, or both, structure 3, FIG. 2A). Further degradation and decomposition of the initial product results in a variety of other substances, including derivatized 2,5-trimethyl-1,3-thiazolidine (structure 4, FIG. 2A), derivatized (2E)-3-[(2-methyl-2-sulfanylpropyl)amino]prop-2-enoic acid (structure 5, FIG. 2B), derivatized 1-amino-2-methylpropane-2-thiol (structure 6, FIG. 2B), derivatized 2,2-dimethyl-2,3,7,7a-tetrahydroimidazo[5, l-b][1,3]thiazole-7-carboxylic acid (structure 7, FIG. 2B), and derivatized (4E)-4-([(2-methyl-2-sulfanylpropyl)amino]methylidene)-1,3-oxazolidin-5-one (structure 8, FIG. 2C). Similarly, β-lactamase-mediated hydrolysis of an early generation cephalosporin with the common 8-oxo-5-thia-1-azabicyclo[4.2.0]oct-2-ene-2-carboxylic acid structure (structure 9, FIG. 2C) results in an initial product, derivatized 2-(carboxymethyl)-3,6-dihydro-2H-1,3-thiazine-4-carboxylic acid (structure 10, FIG. 2C) that further degrades into other compounds including derivatized 2-(carboxymethyl)-5-methylidene-1,3-thiazinane-4-carboxylic acid (structure 11, FIG. 2D).

It will be evident to those of ordinary skill that many possible substances may possess structures that render them adequate substrates for β-lactamases. Such substances include β-lactam antibiotics that are known to be hydrolyzed by β-lactamases as well as other existing or yet-to-be-conceived substances containing the required β-lactam ring. Such substances may contain as the core structure a penam, cepham, penem, cephem, carbapenem, carbacephem, oxapenem, oxacephem, or monobactam. Such substances may be further modified in order to render them more stable in the absence of enzymatic action, or for other purposes. Any substance can be readily recognized as an adequate substrate for a given β-lactamase by contacting the substance with the enzyme in conditions wherein the enzyme is known to be active, and assessing whether hydrolysis products are present after a period of time, by mass spectroscopy or other known methods. Alternatively, a substance can be contacted with β-lactam-resistant bacteria for the same purpose. Any substance determined to be an adequate substrate for a particular set of β-lactamases may be used in the present invention.

The presence of an enzyme may be deduced from detecting the product of a reaction mediated by the enzyme upon a substance known to be an adequate substrate for the enzyme. Many assays are based on this principle, including the nitrocefin assay for β-lactamases. The instant invention uses antibodies, or functional analogs thereof, to detect such products.

In some embodiments of the instant invention, antibodies binding to the products of hydrolysis of β-lactam-containing substrates by β-lactamases are used to determine the presence of β-lactamases in a sample. The general process is to 1) contact the β-lactam-containing substrate with the sample; 2) perform an assay with antibodies, or functional analogs thereof, capable of binding the hydrolysis product of β-lactamase action upon the substrate to determine the presence of the hydrolysis products in the sample following a period of time; 3) correlating the presence of β-lactamase in the sample from the presence of the product; 4) correlating the presence of β-lactam-resistant bacteria in the human or animal or other source of the sample; and 5) selecting specific antibiotics for the treatment of infection in the human or animal, based on the detection or non-detection of β-lactam-resistant bacteria. In some embodiments, the assay provides a yes or no result without quantitation of the amount of the hydrolysis product present. In some embodiments, the assay is quantitative, and the subsequent inferences are based on the level of hydrolysis product detected. In some embodiments, the assay is rapid and performed directly on biological fluid or a tissue sample obtained from a human or animal. In some embodiments, antibodies binding to penicilloic acid-containing hydrolysis products of penicillanic acid-containing substrates are used to determine the presence of any β-lactamases in a sample.

Further differentiation of the type of β-lactamase present in a sample can be achieved by utilizing substrates that are hydrolyzed by one set of β-lactamases but not another set. In these embodiments of the present invention, antibodies specific for the hydrolysis products of such selective substrates are used to determine the presence in a sample of a class of β-lactamases that is capable of hydrolyzing the provided substrate. In some embodiments, the substrate is a third generation cephalosporin, the antibodies bind to one or more products of hydrolysis of the third generation cephalosporin, and the assay is used for the determination of the presence in a sample of β-lactamases of the CTX-M and AmpC classes, some variants of TEM and SHV, and carbapenemases—but not TEM-1, TEM-2, SHV-1, or penicillinases.

Some β-lactamases, the carbapenemases, are capable of hydrolyzing all or almost all β-lactams available today, including the clinically important carbapenems. These enzymes include the New Delhi metalloproteases (NDMs), KPCs, IMPs, VIMs, and several OXA variants. Other common β-lactamases, including TEM, SHV, and CTX-M, do not hydrolyze carbapenems, and pathogens expressing these enzymes remain susceptible to carbapenems. Some embodiments of the present invention use substrates that can be hydrolyzed by carbapenemases, but not by other classes of β-lactamases, to determine the presence of carbapenemases in a sample. These embodiments use antibodies, or functional analogs thereof, to detect the products of carbapenemase hydrolysis of such substrates. In some embodiments, carbapenem-containing substrates possessing the common 7-oxo-1-azabicyclo[32.0]hept-2-ene-2-carboxylic acid structure (structure 12, FIG. 2D) and antibodies binding to hydrolysis products thereof (for example, derivatized 5-(carboxymethyl)-4,5-dihydro-1H-pyrrole-2-carboxylic acid, structure 13, FIG. 2D) are used to determine the presence of any carbapenemases in a sample.

Other embodiments of the present invention can be used to determine if the sample contains (β-lactamases which are insensitive to inhibition by commonly used lactamase inhibitors, including clavulanic acid and sulbactam. If the substrate is contacted with the sample in the presence of a lactamase inhibitor, followed by detection of enzymatic hydrolysis products of the substrate using specific antibodies, then the detection of the products indicates the presence in the sample of β-lactamases insensitive to the inhibitor. In some embodiments, penicillanic acid-containing substrates together with clavulanic acid are contacted with the sample, and antibodies binding to penicilloic acid-containing hydrolysis products of penicillanic acid-containing substrates are used to determine the presence of any β-lactamases in the sample.

Some β-lactamases are constitutively expressed by the bacteria that harbor the genes encoding them. The expression of some β-lactamases is rapidly upregulated in some bacteria after exposure to the β-lactam. In some embodiments, the β-lactam-containing substrate is contacted with the sample for a sufficient period of time to allow increased expression of inducible β-lactamases by bacteria that may be present in the sample.

The most common mechanism of bacterial resistance to aminoglycoside antibiotics is enzymatic modification of the antibiotic. Aminoglycoside modifying enzymes (AMEs) can add phosphate, adenyl, or acetyl groups to hydroxyl and amine groups of various aminoglycosides. AMEs are divided into three functional groups: the N-acetyltransferases (AAC) add an acetyl group to amine groups of aminoglycosides; the O-adenosyltransferases (ANT) add an adenyl group to hydroxyl groups of aminoglycosides; and the O-phosphotransferases (APH) add a phosphate group to hydroxyl groups of aminoglycosides. The described members of each AME group have specific target ranges and site specificity on the aminoglycoside backbone. For example, it is known that at least 10 variants of the enzyme AAC(3) can acetylate gentamicin (structure 14, FIG. 2E) at position N3′ (N3′-acetylgentamicin, structure 15, FIG. 2E). The most prevalent AMEs acetylate aminoglycosides at positions N3′ and N6′.

The possession of at least one AME can confer upon a bacterium resistance to at least some aminoglycosides. It is therefore desirable to rapidly detect AMEs in a sample. In some embodiments of the present invention, a method is provided for the detection of AMEs by 1) contacting a sample suspected of containing AME, or AME-expressing bacteria, with a suitable substrate for at least one AME, and 2) detecting the presence of products of AME action upon the substrate, using antibodies, or analogs thereof, capable of binding to the products. In some embodiments, the substrate is an aminoglycoside antibiotic and the products are members of the set comprising N-acetylated aminoglycosides, O-adenosylated aminoglycosides, and O-phosphorylated aminoglycosides. In some embodiments, the substrate is gentamicin and the product is N-acetylated gentamicin at position N3′ (structure 15, FIG. 2E). The presence in a sample of aminoglycoside-resistant bacteria is correlated from the detection of products of AME action upon a suitable substrate. In some embodiments, methods are provided for the selection of antibiotics for the treatment of a bacterial infection in a human or animal, comprising 1) contacting the sample with a suitable substrate for one or more AMEs; 2) performing an assay, using antibodies, or functional analogs thereof, capable of binding the product of AME action upon the substrate, to detect the presence of such products in the sample following a period of time; 3) correlating the presence of AME in the sample from the presence of the product; 4) correlating the presence of aminoglycoside-resistant bacteria in the human or animal or other source of the sample; and 5) selecting specific antibiotics for the treatment of infection in the human or animal based on the detection or non-detection of aminoglycoside-resistant bacteria. In some embodiments, the assay provides a yes or no result without quantitation of the amount of product present. However, a quantitative assay can be performed, and the subsequent correlations are based on the level of product detected. In some embodiments, the assay is rapid and performed directly on biological fluid or a tissue sample obtained from a human or animal.

Enzymes can also confer antibiotic resistance by mechanisms other than direct deactivation of the antibiotic compound. For example, resistance to the polymyxin class of antibiotics is mediated by enzymes that modify the lipopolysaccharide of bacterial cell walls to introduce amine-containing groups. These modifications, which include ethanolamine, aminoarabinose, and galactosamine, are enzymatically added to the phosphate groups of bacterial lipid A. The amine-modified lipopolysaccharide renders the bacterium resistant to polymyxins, including colistin.

In some embodiments, methods are provided for the detection of enzymes capable of adding amine-containing groups to bacterial lipid A or to bacterial lipopolysaccharide. In some embodiments, antibodies binding to the products of hydrolysis of β-lactam-containing substrates by β-lactamases are used to determine the presence of β-lactamases in a sample. The general process is to 1) contact the sample with a suitable substrate for enzymes capable of adding ethanolamine, aminoarabinose, or galactosamine to bacterial lipid A; 2) perform an assay, comprising antibodies, or functional analogs thereof, capable of binding the product of reactions carried out by at least one enzyme upon the substrate, to determine the presence of such products in the sample following a period of time; 3) correlating the presence of the enzyme in the sample from the presence of the product; 4) correlating the presence of polymyxin-resistant bacteria in the human or animal or other source of the sample; and 5) selecting specific antibiotics for the treatment of infection in a human or animal, based on the detection or non-detection of polymyxin-resistant bacteria. In some embodiments, the assay provides a yes or no result without quantitation of the amount of product present. However, a quantitative assay can be performed and the subsequent correlations are based on the level of product detected. In some embodiments, the assay is rapid and performed directly on biological fluid or a tissue sample obtained from a human or animal.

In some embodiments, the substrate is bacterial lipid A or a fragment thereof. In other embodiments, the substrate is lipid A present in a specific set of bacterial species, and absent in other species. In some embodiments, antibodies binding to phosphoethanolamine-containing products are used to determine the presence of polymyxin-resistant bacteria in a sample. In other embodiments, antibodies binding to phosphoaminoarabinose-containing products are used to determine the presence of polymyxin-resistant bacteria in a sample. In some embodiments, antibodies binding to phosphogalactosamine-containing products are used to determine the presence of polymyxin-resistant bacteria in a sample.

In one embodiment of the invention, an antibody fragment comprises an antigen binding site of the intact antibody and thus retains the ability to bind antigen. In another embodiment, an antibody fragment, for example, one that comprises the Fc region, retains at least one of the biological functions normally associated with the Fc region when present in an intact antibody. For example, such an antibody fragment may comprise an antigen-binding arm linked to a sequence capable of conferring stability to the fragment.

In some embodiments herein, the relevant antigen is a product of enzymatic action upon a particular substrate. It is desirable that an antibody that selectively binds the product does not also bind to the particular substrate. In some embodiments, the antigen comprises penicilloic acid, and the desirable antibodies bind to this antigen but not to substances comprising penicillanic acid. In some embodiments, the antigen comprises acetylated or adenosylated or phosphorylated aminoglycoside, and the desirable antibodies bind to this antigen but not to aminoglycosides lacking the acetyl or adenyl or phosphoryl moieties. In some embodiments, the antigen comprises ethanolamine-modified bacterial lipid A, and the desirable antibodies bind to this antigen but not to lipid A lacking the ethanolamine moiety.

It will be evident to those of skill in the art that other substrates, products, antibodies or analogs thereof, and assay formats may be used to achieve similar results. It will also be evident that other known or newly described enzymes, the presence of which confers upon a bacterium resistance to one or more antibiotics, may be detected by one or more of the disclosed methods with only minor modifications to the basic process.

Aptamers are nucleic acid molecules that may be engineered through repeated rounds of in vitro selection to bind to various targets including antigens. Because of their specificity and binding abilities, aptamers have great potential as diagnostic agents. Methods of development and preparation of aptamers, including those known as SELEX, are known and well-described. Variations on the SELEX process, such as photo-SELEX, counter-SELEX, chemi-SELEX, chimeric-SELEX, blended-SELEX, and automated-SELEX, have also been reported. Any of these methods may be used to prepare aptamers capable of binding to the product of an enzymatic reaction performed by a bacterial enzyme upon a provided substrate, but not binding to the substrate.

For many enzymes that are known to or suspected to confer antibiotic resistance, substrates and reaction products are known. Other substances may also be suitable substrates for such enzymes. Provided are methods of identifying substances which are suitable for use as substrates for bacterial enzymes capable of conferring antibiotic resistance upon a bacterium. These assays may comprise screening of large libraries of candidate substances; alternatively, the assays may be used to focus on particular classes of compounds selected with an eye towards structural attributes that are believed to make them more likely to be suitable substrates for particular enzymes. By screening, it is meant that one may assay a series of candidate substances for the ability to be converted to a different substance by the enzyme of interest.

Screening methods for suitable substrates for particular enzymes are known. For example, a measured quantity of a substance may be contacted with purified enzyme for a period of time, and then the quantity of remaining substance measured again. A reduction in the quantity of the substance indicates that the enzyme has converted some amount of substance into a different substance, and therefore the substance is a suitable substrate for the enzyme. Methods of determining the quantity of starting and remaining substance include mass spectrometry (MS), high performance liquid chromatography (HPLC), gas chromatography (GC), combinations of these methods (GC-MS, LC-MS), and other methods known to those of ordinary skill in the art.

In a similar manner, products of the conversion of a substrate by an enzyme can be identified by known methods. For example, conversion products of a substrate by an enzyme may be separated by HPLC, GC, or other methods, and products identified by analytical methods including MS, nuclear magnetic resonance (NMR), and other known methods.

Also provided are methods of screening antibodies, or functional analogs thereof, for use in the present invention. By screening, it is meant that one may assay a series of candidate substances for 1) the ability to bind a product of the conversion of a suitable substrate by one or more enzymes capable of conferring antibiotic resistance upon a bacterium, and 2) the absence of binding to the suitable substrate. To identify an antibody with this property, one generally will perform one immunoassay using a preparation known to comprise known products of the conversion of a particular substrate by one or more enzymes of interest, and a second immunoassay using a preparation known to comprise the particular substrate.

These immunoassays will further comprise methods to detect the occurrence of binding between a candidate antibody and the preparation. Examples of methods useful in identifying antibodies having bound a target antigen include: ELISA, RIA, CLIA, fluorescence assays, and label-free binding assays wherein unbound antibody is removed by washing steps and only antibodies which have bound to a target remain attached to a solid support. For substrates and reaction products which are of low molecular mass, i.e. <2000 daltons, competitive immunoassays may be suitable. Analogous methods can also be used to identify suitable antibody fragments, including scFv, and aptamers.

Those of skill in the art recognize that other sequential screening methods and other assay formats may also be used to achieve substantially identical results, that mice immunized with other antigens can also be used to produce equivalent monoclonal antibodies, and that these methods can be applied to selecting antibodies binding to products of enzymatic reactions and not to the substrates of the reactions.

The present invention also provides devices that are useful to detect the presence of antibiotic-resistant bacteria a sample. These devices may comprise a surface and at least one agent capable of binding to one or more products of reactions performed upon a substrate by bacterial enzymes. The surface may be any surface to which the desired agents may be attached, including, but not limited to, a microplate or a lateral flow chromatographic test strip. It is contemplated that the device includes a solid support that contains a sample application zone and a capture zone.

The lateral flow chromatographic assay (LFA) is a particular embodiment that allows the user to perform a complete immunoassay within 10 minutes or less. There can be variations of the lateral flow format, including: a variety of porous materials including nitrocellulose, polyvinylidene difluoride, paper, and fiber glass; a variety of test strip housings; colored and fluorescent particles for signal detection including colloidal metals, sols, and polymer latexes; a variety of antibody labels, binding chemistries, and antibody analogs; and other variations. The antibodies can be labeled with, for example, biotin, an enzyme, a latex particle, a metal colloid particle, a fluorescent dye, a quantum dot, or carbon nanotube. Any embodiment of the lateral flow assay may be used for detection of one or more products of reactions performed upon a substrate by bacterial enzymes.

The method described herein may be used to detect β-lactam-resistant bacteria in a wide variety of samples. The method may be used on clinical urine samples from humans or animals suspected of having a UTI, as the sensitivity of detection is adequate for the concentration of bacteria expected in such samples. For many other clinical liquid samples, the concentration of bacteria indicative of infection is much lower—as low as a single bacterium in a sample, as is the case with blood samples from humans or animals suspected of having a bloodstream infection (BSI). For such samples, concentration or expansion of the bacteria may be necessary to obtain a preparation for meaningful analysis by the present embodiment. When it is desirable to detect the presence of β-lactam-resistant bacteria in a clinical or non-clinical sample with a possibly low concentration of bacteria, e.g., <10,000 CFU/ml, the sample may be: 1) concentrated by centrifugation or filtration to reduce the volume while retaining the bacteria in the remaining volume; or 2) cultured to expand the number of bacteria present; or 3) extracted with magnetic particles attached to a bacteria-binding ligand; or 4) other methods that may achieve the same ends. All such processed samples are capable of being analyzed by the method of the invention, without significant changes to the method, with the result informative as to the presence of β-lactam-resistant bacteria in the original sample. A particular advantage of the present method is the low volume of required sample, which allows a simple concentration step to produce a usable sample with adequate sensitivity. Other samples that may be analyzed by the present method, either with or without processing, include, without limitation, any biological fluid, culture medium, beverages, environmental and drinking waters, processed and raw foods with an extractable liquid component, and laboratory samples.

The method can be modified in many ways without deviating from the general premise and effectiveness. For example, other substrates may be used in place of penicillin, including ampicillin, amoxicillin, and other β-lactam-containing substances capable of being hydrolyzed by β-lactamase-harboring bacteria. Any bacteria harboring active β-lactamases, and therefore resistant to β-lactams, can be detected by the method. Other antibodies, reactive with hydrolyzed (β-lactams but not with intact β-lactams, can be used. Other signal-generating moieties, including other enzymes, horseradish peroxidase, fluorescent tags, chemiluminescent moieties, and radioactive labels, can be used. Other formats of competitive and non-competitive immunoassays can also be used to the same effect. The method may be performed by automated and robotic equipment. The method may be employed as part of a microfluidic device or a kit, all without substantial deviations.

### EXAMPLES

### Example 1: Detection of β-Lactam-Resistant Bacteria by ELISA

This example used monoclonal antibodies reactive with benzylpenicilloic acid, a product of β-lactamase mediated hydrolysis of penicillin G, using an ELISA procedure to detect β-lactam-resistant bacteria.

Anti-benzylpenicilloic acid antibody CH2011 and penicilloic acid conjugated to bovine serum albumin (Silver Lake Research Corporation, Azusa, Calif., and Meridian Life Sciences, Memphis, Tenn.) were used to construct an indirect competitive ELISA. Monoclonal antibody CH2011 binds to the BSA-penicilloic acid conjugate and to hydrolyzed penicillin, but not to penicillin.

Samples of β-lactam-resistant Escherichia coli harboring a plasmid expressing the β-lactamase TEM-1 (pET100, ThermoFisher Scientific, Waltham, Mass.) were prepared by diluting an overnight culture of the bacteria in phosphate-buffered saline (PBS) by serial 10-fold dilutions. Parallel samples were prepared in the same manner with control E. coli lacking the plasmid. The parental overnight cultures were enumerated by heterotrophic plate count, and the bacterial concentration of each sample was calculated from the dilution factor. To a 100-microliter volume of each sample was added 5 microliters of freshly prepared penicillin G potassium (Sigma Chemical Company, St. Louis, Mo.) in methanol (1 microgram/milliliter) and allowed to incubate for 30 minutes at room temperature. A volume of 50 microliters of each treated sample was added to duplicate wells of a polystyrene microtiter plate (Costar, Corning, NY) previously coated with penicilloic acid-BSA conjugate in carbonate buffer, pH 9.0, overnight at room temperature, and then washed 4 times with PBS with 0.05% Tween-20 detergent (PBST). Antibody CH2011 was then added to the wells at 50 nanograms/ml, 50 microliters/well, and allowed to react for 30 minutes. The plate was then washed again, and 50 microliters of horseradish peroxidase-conjugated goat anti-mouse-immunoglobulin antiserum (American Qualex, Temecula, Calif.) was added to each well. After washing, SureBlue peroxidase substrate (KPL, Gaithersburg, Md.) was added to each well and incubated in the dark for 10 minutes. The reaction was stopped by addition of 0.1M HCl and the plate was read on a microplate spectrophotometer at 450 nm.

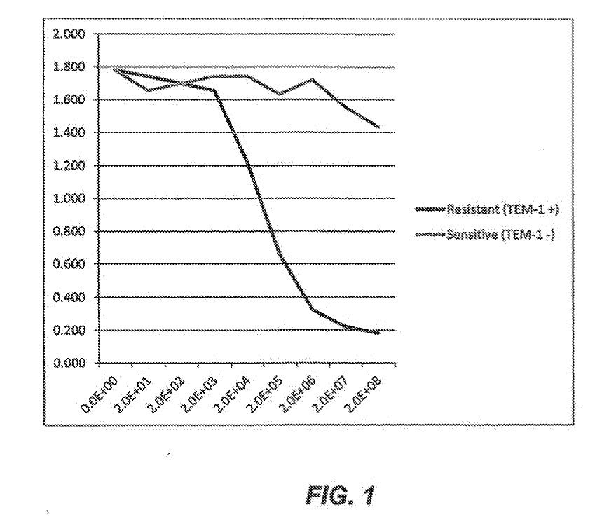

The results are shown in FIG. 1. In the competitive assay, maximum binding of antibody to the penicilloic acid-BSA conjugate occurs when there is no competing hydrolyzed β-lactam in the sample. Increasing concentrations of hydrolyzed β-lactam bind to the antibody, resulting in the commensurate decrease of binding of the antibody to the coated plate.

The results are consistent with the detection by the competitive ELISA of the products of the hydrolysis of penicillin by the TEM-1 lactamase present in E coil harboring the plasmid encoding this enzyme. The control E. coli, lacking the β-lactamase and therefore sensitive to penicillin, did not hydrolyze penicillin. This assay was shown to be capable of detecting β-lactam-resistant E. coli and not β-lactam-sensitive E. coli. The limit of detection of this embodiment of the invention was <10⁴ CFU/ml of 1β-lactam-resistant bacteria.

### Example 2: Detection of 1-Lactam-Resistant Bacteria in Urine Samples with a Lateral-Flow Immunochromatographic Test Kit

Urinary tract infections (UTI) in humans and animals are characterized by the presence in urine of culturable pathogenic bacteria. Most UTIs in humans are caused by Gram-negative bacteria, with E. coli being found in >80% of UTIs and other Enterobacteriaceae responsible for most of the rest. β-lactam-resistant uropathogens are common, with studies reporting ˜25-40% prevalence in the US. In this example, a lateral flow immunoassay was used to detect β-lactam-resistant bacteria directly in urine samples from patients with Gram-negative bacteriuria.

This example uses the particular variation of the lateral flow immunoassay format, the internally referenced competitive immunoassay, described in U.S. Pat. Nos. 6,103,536; 6,287,875; 6,368,875; and 6,649,418, incorporated herein by reference in their entirety. Those skilled in the art are aware of many variations of the lateral flow immunoassay format, any of which may be equivalent to the present example. The preferred method uses MAb CH2011 and BSA conjugated to penicilloic acid, although many other antibodies and paired conjugates may be equivalent.

Colloidal gold was prepared according to published procedures derived from the Turkevich method (J. Turkevich, et al., Discuss. Faraday. Soc., 1951, 11, 55-75; G. Frens, Nature (London). Phys. Sci. 1973, 241:20-22.; Slot, J. W. and H. J. Geuze, Eur. J. Cell Biol. 1985, 38:87-93) and conjugated to purified CH2011 by previously described methods (Oliver C. Methods Mol. Biol. 2010, 588:363). CH2011 gold conjugate was suspended in buffer containing 2 mM sodium borate, pH 9.0, 1% bovine serum albumin, and 0.5% Tween-20 detergent, dispensed into cylindrical flat-bottom test vials (Jade Scientific, Westland, Mich.) and dried. Benzylpenicillin, diluted in methanol to 1 microgram/ml, was dispensed into the same vial and also dried.

Immunochromatographic lateral flow test strips were prepared as previously described (Wong, R C, and Tse H Y. Lateral Flow Immunoassay (New York: Humana Press) 2009; Rapid Lateral Flow Test Strips: Considerations for Product Development (Bedford, Mass.: Millipore Corp., 2008); Weiss, A., IVD Technology, November 1999, p. 48). Test strip chromatographic media included Hi-Flow plastic-backed nitrocellulose membrane (Millipore Corp., Bedford, Mass.), Hi-Flow glass fiber media (Millipore Corp., Bedford, Mass.), acrylic plastic protective cover (G&L, San Jose, Calif.), and adhesive-coated plastic backing (G&L, San Jose, Calif.). BSA-penicilloic acid conjugate was deposited onto the nitrocellulose portion of a lateral flow immunoassay test strip, serving as a “test line” of a typical lateral flow assay format. Polyclonal goat antiserum to mouse immunoglobulins (GAM-Ig; American Qualex, Temecula, Calif.) was deposited at the “control line” of the same nitrocellulose, in a position downstream from the BSA-penicilloic acid conjugate relative to the flow of sample through the strip.

In this example, 250 μl of a urine sample was dispensed in the vial with dried gold conjugate and antibodies, and allowed to incubate for 15 minutes to rehydrate the dried reagents. During this time, penicillin hydrolyzed by β-lactamases contained in the urine sample was bound by the CH2011 antibodies. Following the incubation, the test strip was placed in the vial so that the fiberglass portion was in contact with the sample. Migration of sample through the test strip, driven by wicking action, allows all reagents to come into contact with the test line. Here, any free CH2011 antibodies not already bound to hydrolyzed penicillin can bind to the BSA-penicilloic acid conjugate, thereby immobilizing any gold conjugate particles that comprise such free antibodies. Particles comprising already bound CH2011 bypass the “test line” and can bind to the “control line” downstream. Therefore, the ratio of red color at the test line and the control line can be used as an indication of the concentration of hydrolyzed penicillin in the sample. In this method, if the test line is visibly darker than the control line, the test result is “negative”; if the lines are equally dark, or if the control line is darker than the test line, the result is “positive.” The results were determined visually 10 minutes after placing the test strip in the vial.

The test can alternatively be done by using a traditional competitive lateral flow immunoassay test strip format. For this, purified antibody CH2011 was directly coated onto colloidal gold particles in accordance with published procedures, dispensed onto the fiberglass pad section of immunochromatographic test strips, prepared as above. BSA-penicilloic acid conjugate was deposited at the test line of the lateral flow test strip membrane, and GAM-Ig was deposited on the control line. The test procedure is as above, but the result is interpreted as “negative” if there is any color at the test line and “positive” if there is no color at the test line.

The traditional competitive lateral flow immunoassay test strip format was used to assay normal urine spiked with varying concentrations off coil, the pathogen responsible for most urinary tract infections worldwide. Sterile-filtered urine from normal donors was spiked with cultured E. coli with and without a plasmid containing the bla gene, encoding the TEM-1 β-lactamase (pET100, ThermoFisher Scientific, Waltham, Mass.). Bacteria with this plasmid are resistant to penicillin, ampicillin, and many other β-lactam antibiotics. Samples were tested by the method described in this example, and the bacteria enumerated by heterotrophic plate count. The results are shown in Table 1.

TABLE 1
Results of Testing Urine Samples with Lateral Flow Immunoassay
Test Strips with CH2011 Antibodies

| | E. coli TEM-1 | CULTURE | LATERAL |
|---|---|---|---|
| | (Resistant/ | RESULT | FLOW TEST |
| SAMPLE | Sensitive) | (CFU/ml) | RESULTS |
| 1 | No Bacteria | 0 | Negative |
| 2 | Resistant | 1 × 10² | Negative |
| 3 | Resistant | 4 × 10² | Negative |
| 4 | Resistant | 6 × 10³ | Negative |
| 5 | Resistant | 3 × 10⁴ | Positive |
| 6 | Resistant | 2 × 10⁴ | Positive |
| 7 | Resistant | 2 × 10⁵ | Positive |
| 8 | Resistant | 4 × 10⁵ | Positive |
| 9 | Resistant | 1 × 10⁵ | Positive |
| 10 | Resistant | 8 × 10⁵ | Positive |
| 11 | Sensitive | 7 × 10⁴ | Negative |
| 12 | Sensitive | 4 × 10⁴ | Negative |
| 13 | Sensitive | 7 × 10⁵ | Negative |
| 14 | Sensitive | 8 × 10⁶ | Negative |

The results indicate that the competitive lateral flow immunoassay test strip format is capable of detecting β-lactam-resistant E. coli in urine with an apparent limit of detection of ˜1×10⁴ CFU/ml. This concentration of bacteria is present in urine samples from typical UTI patients, and therefore the method can be used directly on patient urine samples without any sample preparation steps. This method is specific for the presence of β-lactam resistance, as sensitive bacteria are not detected even at concentrations in excess of typical bacteriuria. The total time-to-result of this method is under 30 minutes. Therefore, this embodiment is a valid and valuable test for the presence of β-lactam-resistant bacteria in urine.

### Example 3: Detection of the Presence of Bacteria and of β-Lactam-Resistant Bacteria in Urine Samples with Two Immunoassay Tests

UTIs in humans and animals are commonly treated empirically, without confirming the presence of bacteria in urine. In many cases of suspected UTIs, urine cultures are negative—no uropathogens are identified. In such cases, a single assay for antibiotic resistance, such as that described in Example 2, would give a negative result, but antibiotic treatment may not be warranted. In this example, a second rapid lateral flow immunochromatographic test is used on the same urine sample to determine whether uropathogenic bacteria are present at clinically significant levels.

This second lateral flow assay detecting significant bacteriuria is known. For example, the RapidBac™ test kit (Silver Lake Research Corporation, Azusa, Calif.) is an immunoassay that detects Gram-negative uropathogens, which represent >90% of all uropathogens, using biomarkers described in U.S. Pat. No. 9,052,314, which is incorporated herein by reference in its entirety. The immunoassay has been shown to have a sensitivity of 96% for Gram-negative uropathogens at a level of 10⁴ CFU/ml. Together, the results of the lateral flow assay of Example 2 and the RapidBac™ test for bacteriuria give a more complete indication of whether antibiotic treatment is warranted, and, if so, which antibiotic to use. The interpretation of the results is shown in Table 2.

TABLE 2
Results of Testing Urine Samples with Two Lateral
Flow Immunoassays

| | Result of | |
|---|---|---|
| Result of | Beta-lactam | |
| Bacteriuria Test | Resistance Test | Indication |
| Negative | Negative | No significant bacteriuria; antibiotic |
| | | treatment not warranted |
| Positive | Negative | Probable UTI; Beta-lactams |
| | | probably effective |
| Positive | Positive | Probable UTI; Beta-lactams likely |
| | | ineffective |

### Example 4: Detection of Bacteria Resistant to Specific β-Lactams—3^{rd} Generation Cephalosporins

The present invention can be used to detect bacteria resistant to third-generation cephalosporins. The provided substrate can be a third-generation cephalosporin, including, for example, cefotaxime, and the antibodies, or analogs thereof, which bind to hydrolyzed cefotaxime but not to the parent cefotaxime. A competitive immunoassay can be used to detect hydrolyzed cefotaxime following a 10-60 minute incubation of the sample with cefotaxime. The results of the immunoassay are interpreted as follows: positive (hydrolyzed cefotaxime detected) indicate the presence in the sample of bacteria resistant to third-generation cephalosporins, and a negative result (no hydrolyzed cefotaxime detected) indicates the absence of such bacteria in the sample. Because enzymes capable of hydrolyzing third-generation cephalosporins are typically also capable of hydrolyzing penicillins and first- and second-generation cephalosporins, the results are also indicative of resistance to these antibiotics.

### Example 5: Detection of Bacteria Resistant to Specific β-Lactams—Carbapenems

The present invention can also detect bacteria resistant to carbapenems. Because of the increasing prevalence of multi-drug-resistant bacteria worldwide, this class of beta-lactams may be the last-line antibiotic in the most serious cases, and resistance to the class may necessitate extra measures to isolate the patient and prevent further spread of the pathogen. Furthermore, pathogens carrying carbapenemases often possess resistance to many other classes of antibiotics, making the timely identification of carbapenem-resistant pathogens important.

In this Example, the provided substrate was a carbapenem, imipenem, and the antibodies, or analogs thereof, which bind to hydrolyzed carbapenem but not to the parent carbapenem. A competitive immunoassay can be used to detect hydrolyzed carbapenem following a 10-60 minute incubation of the sample with the carbapenem substrate. The results of the immunoassay are interpreted as follows: positive (hydrolyzed carbapenem detected) indicate the presence in the sample of bacteria resistant to carbapenems, and a negative result (no hydrolyzed carbapenem detected) indicate the absence of such bacteria in the sample. Because enzymes capable of hydrolyzing carbapenems are typically also capable of hydrolyzing all other beta-lactams, the results are also indicative of resistance to these antibiotics.

### Example 6: Detection of Bacteria Resistant to β-Lactams and Resistant to Suppression of Resistance by Lactamase Inhibitors

Inhibitors of beta-lactamases are known, including clavulanic acid and sulbactam, and are commonly used in combination with beta-lactam antibiotics to suppress the resistance of bacteria to the antibiotics. For example, clavulanic acid can be combined with amoxicillin or ticarcillin to enable activity of these antibiotics. However, many beta-lactamases cannot be inhibited by such inhibitors—clavulanic acid does not inhibit CTX-M beta-lactamases and sulbactam does not inhibit AmpC cephalosporinases. The present invention can be used to determine the presence in a sample of bacteria resistant to at least some beta-lactam antibiotics and also insensitive to the inhibition of resistance by one or more beta-lactamase inhibitors. In this Example, the provided beta-lactamase substrate can be combined with one or more beta-lactamase inhibitors, for example, amoxicillin and clavulanic acid. The antibodies, or analogs thereof, bind to hydrolyzed beta-lactam product but not to the parent substrate. A competitive immunoassay can be used to detect hydrolyzed beta-lactam following a 10-60 minute incubation of the sample with the beta-lactam and the beta-lactamase inhibitor. The results of the immunoassay are interpreted as follows: positive (hydrolyzed beta-lactam detected) indicates the presence in the sample of bacteria resistant to at least some beta-lactam and insensitive to inhibition of resistance by the provided inhibitor, and a negative result (no hydrolyzed beta-lactam detected) indicates that such bacteria have not been detected in the sample.

### Example 7: Detection of Bacteria Resistant to Aminoglycoside Antibiotics

The present invention can be used to determine the presence in a sample of bacteria resistant to antibiotics, in this case aminoglycosides. This class of antibiotics is useful for the treatment of many bacterial infections in humans and animals, especially Pseudomonas aeruginosa infections, and is becoming more important as resistance to other classes of antibiotics is increasing. Because much of the resistance to aminoglycosides is due to the presence in bacteria of aminoglycoside-modifying enzymes, this resistance can be detected by embodiments of the present invention that detect the presence of AMEs in a sample.

In this example, the provided substrate is an aminoglycoside, such as, for example, gentamicin, and the antibodies, or analogs thereof, bind to one or more members of the set comprising acetylated aminoglycosides, adenosylated aminoglycosides, and phosphorylated aminoglycosides, but not to the substrate aminoglycoside. The presence of aminoglycoside-resistant bacteria in a sample comprises (a) contacting the sample with gentamicin, (b) incubating the sample for 10-60 minutes to allow acetylation of gentamicin by AAC(3)-type AMEs that may be present in the sample or phosphorylation of gentamicin by APH(2″)-type AMEs, (c) detecting the presence of 3-acetyl-gentamicin or 2″-phosphogentamicin by a competitive lateral flow immunochromatographic assay comprising antibodies capable of binding to 3-acetyl-gentamicin and 2″-phosphogentamicin, but not to gentamicin. The results of the immunoassay can be interpreted as follows: positive (3-acetyl-gentamicin or 2″-phosphogentamicin detected) indicates the presence in the sample of bacteria resistant to aminoglycosides, and a negative result (no 3-acetyl-gentamicin or 2″-phosphogentamicin detected) indicates that such bacteria have not been detected in the sample. It would be evident to those of skill in the art that other substrates and product-specific antibodies may be used for a substantially similar purpose, for example, if the detection assay is to be used for pathogens with a specific AME expression profile or if local prevalence of specific types of AMEs is known to be high.

### Example 8: Detection of Bacteria Resistant to Polymyxin Antibiotics

The present invention can also be used to determine the presence of bacteria that are resistant to antibiotics in a sample, where the mechanism of resistance is dependent on enzymes capable of modifying the molecular target of antibiotic action. One example of such resistance is the bacterial resistance to the polymyxin class of antibiotics, where enzymatic addition of amine-containing moieties to bacterial lipid A results in resistance to the polymyxins, notably to the antibiotic colistin. Resistance to this class of antibiotics can be detected by determining the presence in a sample of enzymes that are capable of adding amine-containing moieties to bacterial lipid A.

Bacterial lipid A is a membrane-proximal component of bacterial lipopolysaccharide (LPS). While the membrane-distal elements of LPS vary greatly in their chemical structure between bacterial strains and species, lipid A is relatively homologous between bacterial strains and related species. Therefore, enzymes capable of modifying lipid A may use as a substrate a plurality of divergent LPS structures, fragments comprising lipid A, or lipid A. These substances may be purified from bacterial cultures using known methods.

Enzymes capable of adding amine-containing moieties to bacterial lipid A are known, and others are likely to be discovered in the future. LPS from polymyxin-resistant bacteria has been shown to contain one or more members of the set ethanolamine, aminoarabinose, and galactosamine, as additions to the phosphoryl groups of lipid A.

The provided substrate can be LPS or a fragment thereof, containing bacterial lipid A. The antibodies, or analogs, bind to one or more of phosphoethanolamine-lipid A, phosphoaminoarabinose-lipid A, and phosphogalactosamine-lipid A, but not to the substrate. For example, the presence of polymyxin-resistant bacteria in a sample comprises the steps of (a) contacting the sample with bacterial LPS, (b) incubating the sample for 10-60 minutes to allow addition of ethanolamine to LPS by bacterial enzymes that may be present in the sample, (c) detecting the presence of phosphoethanolamine-LPS by a competitive lateral flow immunochromatographic assay comprising antibodies capable of binding to phosphoethanolamine-lipid A, but not to lipid A. The results of the immunoassay can be interpreted as follows: a positive result (phosphoethanolamine-lipid A detected) indicates the presence in the sample of bacteria resistant to polymyxins, and a negative result (no phosphoethanolamine-lipid A detected) indicates that such bacteria have not been detected in the sample. It would be evident to those of skill in the art that other substrates and product-specific antibodies may be used for a substantially similar purpose.

### Example 9: Detection of Antibiotic-Resistant Bacteria in Stool, Sputum, and Tissue Samples

Examples 1 and 2 describe methods which are used to test liquid samples. The present invention can also be used for detection of antibiotic-resistant bacteria in solid or semi-solid samples, including stool, sputum, tissue, and other non-liquid matrices. In this Example, a sampling method is provided that is useful in making such samples compatible with the general methods for testing liquid samples.

In this example, an absorbent swab is inserted into a solid or semi-solid sample and allowed to absorb any solid or liquid that may attach to the swab. Absorbent swabs used in this manner are known, for example, sterilized cotton swabs from Puritan Medical, Inc., Guilford, Me., USA. The swab is then inserted into a buffer or liquid to allow the absorbed material to partially or totally solubilize in the buffer or liquid. The buffer or liquid is then assayed with the methods described herein to determine the presence of antibiotic-resistant bacteria.

### Example 10: Detection of Antibiotic-Resistant Bacteria in Cultures

Many infections in humans and animals are diagnosed on the basis of low bacterial counts in the relevant biological samples. Examples include bloodstream infections (BSI), in which a positive diagnostic result can be as low as a single detected bacterium in the entire collected volume of blood, as detected by blood culture over several days, and bacterial meningitis, in which a similarly minimal level of detection is used in cultures of cerebrospinal fluid. In such infections, an unprocessed sample cannot be analyzed directly for the presence of antibiotic-resistant bacteria by the methods of other Examples herein—the amount of enzyme present in such samples is simply too small to convert sufficient substrate in a reasonable time. For such infections, the current invention can include a step which expands the number of bacteria that may be present in a sample by culturing the sample in appropriate conditions that promote the growth of the pathogens. After such culture, the culture medium may be tested by the methods of other Examples described herein, with the result indicative of the presence of antibiotic-resistant bacteria in the original sample.

### Example 11: Detection of Antibiotic-Resistant Bacteria in Liquid Samples with Additional Incubation for Slow-Acting Enzymes

Some beta-lactamases are not expressed constitutively, but are activated in the presence of beta-lactam antibiotics. Some bacteria may express only low levels of beta-lactamases, which nevertheless may render these bacteria resistant to beta-lactam antibiotics. Similarly, some AMEs may be slow in their modification of aminoglycoside antibiotics. Enzymes mediating the addition of ethanolamine or other amine-containing groups to bacterial lipopolysaccharides may also be slow in their conversion of a particular presented substrate. In this example, a lateral flow immunoassay is used to detect antibiotic-resistant bacteria in liquid samples, with a procedural modification to enable adequate conversion of a substrate by slow-acting enzymes.

The time required to convert a substrate into a detectable amount of product can be determined experimentally. For example, a lactamase substrate, e.g., ampicillin, can be incubated with bacteria possessing an inducible beta-lactamase of the AmpC class for increasing time periods from 0 min to 2 hrs. The amount of penicilloic acid produced by AmpC cleavage of the penicillin substrate can be determined by the ELISA described in Example 1. The result can be used to establish the time required to incubate the ampicillin substrate in the procedure of Example 2, using a lateral flow chromatographic test to detect the penicilloic acid product following the incubation period. This procedure can be applied directly to urine samples in areas where the prevalence of inducible beta-lactamases in uropathogens is suspected to be high. This procedure can also be applied to samples of liquid bacterial cultures prepared from samples of blood, stool, or tissue swabs from humans or animals suspected of having bacterial infection with inducible AmpC-class beta-lactamases, or other types of bacterial infections which may express lactamases that are slow-acting yet cause resistance.

Similarly, the analogous time period for incubation of samples with a substrate can be experimentally established for aminoglycoside substrates and AME-possessing bacteria and lipid A or lipopolysaccharide substrates and colistin-resistant bacteria. For all of these instances, the determined incubation time can be incorporated into the assay procedure using ELISA, or lateral flow immunochromatographic tests, or other types of assays, using any appropriate substrate for any enzyme capable of rendering a bacterium resistant to a set of antibiotics.

### Example 12: Selection of Antibiotics for Treatment of Suspected Bacterial Infections Based on Rapid Testing for Antibiotic Resistance

In many instances, bacterial infections in humans and animals am treated with antibiotics empirically, without prior diagnostic testing of antibiotic resistance. In most cases, patient symptoms give some indication of the possible bacterial infection and the concomitant need for antibiotic treatment. Similarly, typical bacterial pathogens responsible for the suspected infection are known, as well as the antibiotics that may be effective treatments for the pathogens. However, with current methods, it is difficult to determine whether the specific bacterial pathogen in a particular case is or is not resistant to any of the preferred first-line or second-line or other antibiotics. In the present invention, methods are provided for selection of antibiotics for treatment on the basis of testing for the presence of antibiotic-resistant bacteria in samples from humans or animals suspected of having a bacterial infection.

In this Example, the selection method for the appropriate antibiotic for a given bacterial infection comprises (a) collecting a sample from the human or animal suspected of having a bacterial infection, wherein the presence of infection can be determined from the presence of bacteria in the sample, (b) testing for the presence of antibiotic-resistant bacteria in the sample by any of the methods provided in any of Examples 1-11, (c) determining whether or not antibiotic-resistant bacteria have been detected by the method, (d) if resistance to a class of antibiotics has not been detected, selecting an antibiotic for treatment of the infection from the class of antibiotics; or, if resistance to a class of antibiotics has been detected, selecting an antibiotic for treatment of the infection from another class of antibiotics.

A rapid lateral flow immunochromatographic test, as described in Example 2, can be used to select antibiotics for the treatment of urinary tract infections in humans or animals. A sample of urine of >1 mL is collected from the human or animal suspected of having a urinary tract infection. The sample is assayed for bacteria resistant to beta-lactams by the method described in Example 2. If the result of the lateral flow test is negative, as in Table 1, the antibiotic selected for treatment of the presumed UTI is amoxicillin. If the result of the lateral flow test is positive, the antibiotic selected for treatment of the presumed UTI is ciprofloxacin.

### Example 13: Selection of Antibiotics for Treatment of Suspected Urinary Tract Infections Based on Rapid Testing for the Presence of Bacteria and for Antibiotic Resistance

In the case of UTIs, as in many other types of bacterial infections, suspected bacterial infections in humans and animals are treated with antibiotics without prior diagnostic testing of the presence of bacteria. In some instances, patient symptoms may be due to a bacterial infection or to another cause, not amenable to antibiotic treatment. It is desirable in such cases to determine the presence of bacteria in a patient sample, wherein the presence of bacteria in the sample indicates the presence of bacterial infection. In the present invention, methods are provided for selection of antibiotics for treatment on the basis of testing for both the presence of bacteria and for the presence of antibiotic-resistant bacteria in samples from humans or animals suspected of having a bacterial infection.

In this Example, the selection method for the appropriate antibiotic for a given bacterial infection comprises the steps of (a) collecting a sample from the human or animal suspected of having a bacterial infection, wherein the presence of infection can be determined from the presence of bacteria in the sample, (b) testing for the presence of bacteria in the sample. (c) testing for the presence of antibiotic-resistant bacteria in the sample by any of the methods provided in any Example herein, (d) determining whether or not antibiotic-resistant bacteria have been detected by the method, and (e) using the following method to select an antibiotic, if any, for treatment: 1) if no bacteria have been detected, do not use antibiotics and investigate another explanation for the symptoms; 2) if bacteria are detected, and resistance to a class of antibiotics has not been detected, select an antibiotic for treatment of the infection from the class of antibiotics; and 3) if bacteria are detected, and resistance to a class of antibiotics has been detected, selecting an antibiotic for treatment from another class of antibiotics.

Two rapid lateral flow immunochromatographic tests, as described in Example 3, can be used to select antibiotics for the treatment of urinary tract infections in humans or animals. A sample of urine of >1 mL is collected from the human or animal suspected of having a urinary tract infection. The sample is assayed for bacteria and for bacteria resistant to beta-lactams by the method described in Example 3. If the result of the lateral flow test for bacteria is negative, as in Table 2, do not use antibiotics. If the result of the lateral flow test for bacteria is positive and the result of the lateral flow test for beta-lactam resistance is negative, as in Table 2, the antibiotic selected for treatment of the presumed UTI is amoxicillin. If the result of the lateral flow test for bacteria is positive and the result of the lateral flow test for beta-lactam resistance is positive, the antibiotic selected for treatment of the presumed UTI is ciprofloxacin.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure. All references cited herein are incorporated by reference in their entirety.

What is claimed is:

1. A method of detecting the presence of antibiotic-resistant bacteria in a sample, comprising:
(a) contacting the sample with a substrate for one or more bacterial enzymes, wherein the bacterial enzymes are capable of conferring antibiotic resistance upon bacteria possessing the enzymes; and
(b) using one or more antibodies, antibody fragments, or aptamers to detect the presence of one or more products of enzymatic reactions carried out by the bacterial enzymes upon the substance;
wherein the detection of at least one or more products of enzymatic reactions indicates the presence in the sample of bacteria resistant to one or more antibiotics.

2. The method of claim 1, wherein the one or more bacterial enzymes are β-lactamases.

3. The method of claim 1, wherein the bacterial enzymes comprises carbapenemase.

4. The method of claim 1, wherein the substrate is one or more molecules comprising a β-lactam moiety.

5. The method of claim 1, wherein the substrate is one or more molecules containing a carbapenem moiety.

6. The method of claim 1, wherein, prior to (a), the sample is treated with one or more substances.

7. The method of claim 6 wherein the substance comprises a detergent, a buffer, or metal salts.

8. The method of claim 1, wherein the one or more antibodies, antibody fragments, or aptamers are immobilized on a solid support.

9. The method of claim 8 wherein the solid support comprises a particle, a bead, a plastic surface, a glass surface, a porous membrane, an array, or a chip.

10. The method of claim 1, wherein the bacterial enzymes comprise one or more members of the group comprising N-acetyltransferases, O-adenosyletransferases, and O-phosphotransferases, and the substance is one or more molecules containing amino-sugars capable of being acetylated by bacterial N-acetyltransferases or one or more molecules containing sugars capable of being adenosylated or phosphorylated by bacterial O-adenosyletransferases or O-phosphotransferases.

11. The method of claim 1, wherein the bacterial enzymes are capable of adding ethanolamine, aminoarabinose, or galactosamine to bacterial lipopolysaccharides or to bacterial lipid A.

12. The method of claim 1, wherein the antibody is polyclonal or monoclonal.

13. The method of claim 1, wherein the sample is a diluted or non-diluted sample of a group comprising urine, blood, serum, blood products, plasma, saliva, bodily fluid, water, culture medium, petroleum product, fuel, liquid undergoing fermentation, or a beverage.

14. The method of claim 1, wherein the sample is human or animal tissue, stool, sputum, expectorate, an agricultural product, food, solids collected by centrifugation or filtration, soil, or sediment.

15. The method of claim 1, wherein the detecting is performed by a competitive immunoassay, an enzyme-linked immunosorbent assay (ELISA), an immunofluorescence assay (IFA), a radioimmunoassay (RIA), a chemiluminescence immunoassay (CLIA), a lateral flow chromatographic test, or a dot blot, a chromatographic test, a Western blot, an immunoprecipitation assay, or a lateral flow immunoassay device.

16. The method of claim 1, wherein the antibodies, antibody fragments, or aptamers are labeled.

17. A kit for detecting the presence of bacteria resistant to one or more antibiotics in a sample, wherein the kit comprises an antibody, an antibody fragment, or aptamer capable of binding one or more products of enzymatic reactions carried out by bacterial enzymes upon a substance capable of being a substrate of the bacterial enzymes, wherein the bacterial enzymes are capable of conferring antibiotic resistance upon the bacteria possessing the enzymes.

18. The kit of claim 17, further comprising a lateral flow chromatographic assay.

19. The kit of claim 17, further comprising a negative control, a positive control, or both a negative and positive control.

20. A method of treating bacterial infection in an individual comprising:
a) performing the method of claim 1 on a sample from the individual, resulting in detection of antibiotic resistance in the sample, and
b) selecting an antibiotic other than the set of antibiotics capable of being a substrate for the bacterial enzymes for which the substance used in the method is a substrate, thereby treating the bacterial infection in the individual.
